(19) United States
(12) Patent Application Publication
(10) Pub. No.: US 2017/0224974 A1
(43) Pub. Date: Aug. 10, 2017

(54) MEDICAL DEVICE ADAPTER WITH LUER LOCK OUTPUT FITTING

(71) Applicant: AMT Pte Ltd, Singapore (SG)

(72) Inventor: Martin Wong, Singapore (SG)

(73) Assignee: AMT Pte Ltd, Singapore (SG)

(21) Appl. No.: 15/019,658

(22) Filed: Feb. 9, 2016

(51) Int. Cl. A61M 39/10

(52) U.S. Cl. CPC A61M 39/10; A61M 2039/1027; A61M 2039/1016; A61M 39/12

(57) ABSTRACT

A connector for medical devices (e.g., a syringe) allows tubing to be securely connected to a medical device output. Medical devices have male Luer lock ports for tubing connection. Tubing has a female Luer member with conical threads for leak-tight connection to a threaded male Luer member. Here, a first tubular connector member extends from a medical device output for axial fluid flow. A first connector body has a flange rearward of the outward end, and a nose end that tapers radially inwardly toward the outward end. A second tubular connector member is coaxial with the first connector member. An axially divergent portion of the second connector acts as a sheath for the nose end of the first connector, with a first end surrounding a portion of the first connector and with a second flange forward of the first end abutting the first flange of the first connector. The second flange has latch members with flexing endwise teeth that rearwardly project through holes in the first flange, whereby the two connectors latch securely.

### TECHNICAL FIELD

The invention relates to fluid transfer devices for medical equipment and, in particular, to an adapter that interfaces a medical device, particularly a syringe, to a fluid conduit such as medical tubing.

### BACKGROUND ART

U.S. published application US 2002/0173753 describes a fluid transfer adapter for use with a syringe barrel having an open proximal end including a collar having an adapter engaging structure and a distally-facing frusto-conically shaped surface. The adapter has a hub having a proximally-facing frusto-conically shaped surface for mating with a frusto-conically shaped surface on the barrel, with a structure for threadably engaging the hub to the collar of the barrel so that the frusto-conically shaped surface of the hub contacts the frusto-conically shaped surface of the barrel to prevent liquid flow between the surfaces. There is fluid transfer using a passageway therethrough, connected to the distal end of the hub so that a lumen is in fluid communication with a conduit in the hub.

In U.S. Pat. No. 6,969,375 there is a Luer lock type connection device for medical use having a nozzle in which an axial internal passage is formed. The nozzle includes a bearing connection surface and a proximal annular retention zone. The connection device also has a flange forming an axial open seat having a wall that includes a distal part with internal screw thread, and a transverse proximal part in which a central proximal retention opening is formed, and whose diameter is matched to that of the proximal annular zone of the nozzle. The proximal annular retention zone of the nozzle and/or the central proximal retention opening of the flange include a circular band or cord covered or treated with a roughening agent.

In U.S. Pat. No. 4,927,417 there is an adapter for mounting a protective sleeve or needle guard on a syringe or other needle fitting, including a first locking portion for engagement with a locking portion at an end of a syringe, a needle mount at an opposite side of the adapter, and engaging means for selectively engaging the protective sleeve or needle guard into at least a needle protecting position. The needle mount can be separable from the adapter, or can be integrally formed therewith.

In U.S. Pat. No. 4,294,250 a Luer lock connection device for medical use is described of the type that can be used, for example, in continuous ambulatory peritoneal dialysis in which a solution container is coupled via flexible tubing to a patient's tube that communicates with the patient's peritoneal cavity. The Luer lock connection device includes a cooperating male Luer lock connector and a female Luer lock connector, with visual indicating means provided to indicate that the male and female connectors are assembled properly for use when they are in a predetermined relationship. The male and female Luer lock connectors are structured to provide a double sealing arrangement for aiding in maintaining an uncontaminated connection.

Luer lugs for mating with conical Luer lock fittings are known from International Standard ISO 594-2. variants A and B.

A problem is that a threaded connection takes time to disconnect and, at times such as in surgery, time is of the essence. Therefore, a safe and secure quick connect and disconnect adapter for tubing is needed.

### SUMMARY OF INVENTION

The invention is an adapter that allows medical tubing to be securely and quickly connected to an output side of medical device, such as a syringe, using a quick-connect and disconnect adapter. Medical devices often have male Luer lock ports for tubing connection. Tubing having a female Luer member usually has conical threads for secure leak tight connection to a threaded male Luer member. The invention features 3 connector members: a first male part extending from a medical device, a second female part that latches onto the first part in a manner such that the first and second connector members cannot be separated without latch release and a third deformable pivot ring part that releases latching of the first and second parts.

### BRIEF DESCRIPTION OF DRAWINGS

FIG. 1 is a side plan view of a syringe with three ports having an adapter of the invention mounted on each port.

FIG. 2 is a front perspective view of an adapter shown in FIG. 1.

FIG. 3 is a side sectional view of the adapter shown in FIG. 2

FIG. 4 is a front plan view of a variant Luer output connector for the adapter shown in FIG. 3.

FIG. 5 is a rear perspective view of a latching portion of the adapter shown in FIG. 3.

FIG. 6 is an operational view illustrating a release of latching mechanism for the adapter shown in FIG. 3.

FIGS. 7 and 8 are schematic views of two possible connection scenarios for use with a patient.

### DETAILED DESCRIPTION

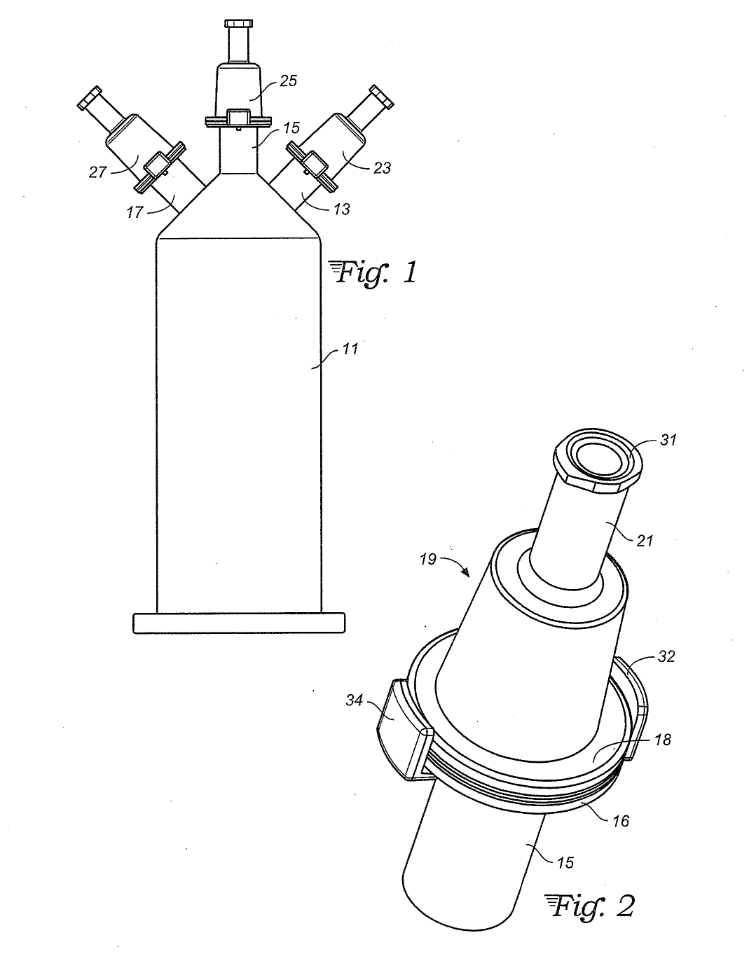

With reference to FIG. 1, a syringe 11 is shown having 3 output ports 13, 15, 17, each fitted with an adapter 23, 25, 27 for connection to tubing, not shown. The output ports are male ports that require adapters to work with Luer lock fittings on medical tubing.

With reference to FIG. 2, the male port 15 is a tubular first connector member of the invention, the port being associated with a medical device, such as the syringe of FIG. 1. The medical device is not limited to a syringe, but could be other devices found in medical, clinical, veterinary, chemical and research laboratories.

The first connector member has a first flange 16, acting as a seat for a second flange 18, part of a second connector member 19 that is a sheath for the nose end of the first connector member and equipped with a flanged tubular male outlet 21 having a central axis and a Luer outlet fitting 31. Although not shown in FIG. 2, the first tubular connector member extends forwardly beyond the first flange 16 in a tubular nose portion 55 that extends through the center of the second flange 16 within the body of the second connector member 19 with axial symmetry and an axial passageway for fluid flow through the tubular nose portion 55 into the tubular male outlet 21.

Note that the first flange 16, although part of the medical device, is a connector member associated with the invention. The first and second flanges are releaseably locked together as will be described below. A third connector member is a deformable ring, not shown, surrounding the outside of the tubular first connector member. The third connector member has opposed extenders 32 and 34, described more fully below, that serve as release members for the locked flanges.

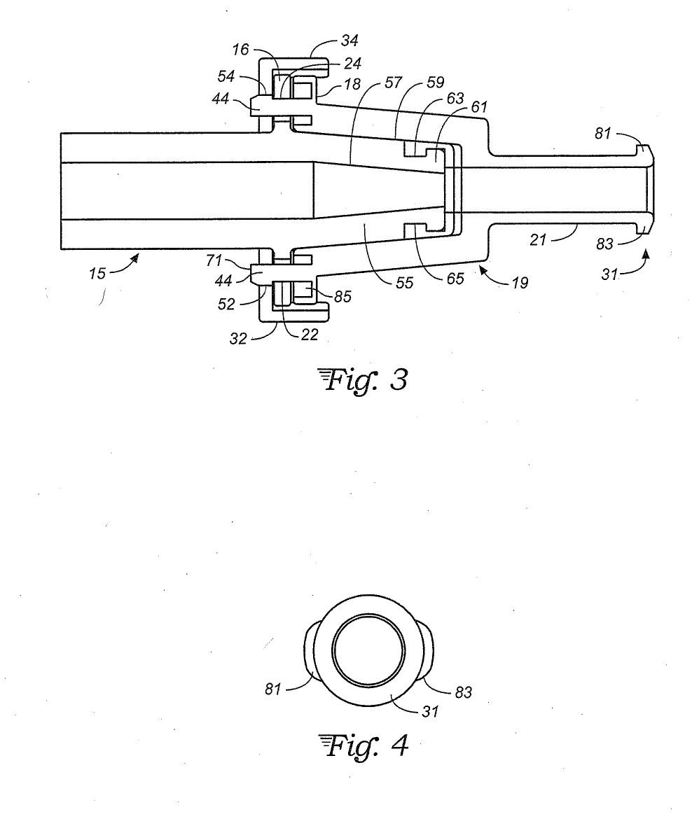

With reference to FIG. 3, the flanged male port 15 of a medical device has, as an integral portion, a first flange 16 with diametrically opposed holes 22 and 24 for latches and diametrically opposed pivot holes 52 and 54 aligned with the latch holes. The tubular male port of the first connector member 15 has a nose portion 55 forward of the second flange 18 with tapering inner and outer diameters 57 and 59 respectively, towards a forward end 61, away from the medical device, with a circumferential detent 63 distal to the second flange 18 in the outer diameter 59 of the tapered nose portion 55. A safety cap 65 with an inward rim in the detent 63 has a central axial hole equal in size to the passageway though the male outlet 21. The safety cap 65 is lightly adhered to the second connector member 19. The circumferential rim becomes lodged in the detent 63 and when the second connector member is separated from the first connector member, the safety cap 65 remains in place unless physically removed. In this manner, only the second connector member may be reconnected to the first connector member. Another second connector member, carrying its own safety cap cannot be put into place joining the first connector member unless the original safety cap is removed because of interference between the original safety cap, seated in the detent, and another safety cap of the other second connector member.

The first connector member having the male port 15 mates with a second tubular female connector member 19, serving as a partial sheath for the first member. In other words, the male port 15 has a forward portion that projects into the second tubular female connector member in a coaxial manner with an axial flow path that provides a continuous conduit for flow material from the first connector member into the second.

The second connector member has projecting latch members 42 and 44 extending rearwardly from the second flange 18, parallel to the axis, and into locking engagement with the opposed pivot holes 52 and 54 in the first flange 16 of the tubular male outlet port 15. The latch members have radially outwardly extending teeth at their ends, i.e. endwise teeth, that flex and prevent separation of the first and second connector members once they are joined. The latch members are slightly flexible so that the flexing teeth can fit through the holes 52 and 54 in the first flange 16, but once through the holes, the latch members flex in a manner so that the teeth at ends of the latch members prevent a reverse path that would result in separation of the first and second connector members by pulling on the two members, unless there is a physical release of the latches with external radial force flexing the teeth so that they will pass back through the holes 52 and 54. An optional pair of concentric washers 85 can surround the latch members 42 and 44 from positions recessed within the second flange 18 at radial distances on either side of the latch members.

From an annular axial zone 71 within the flow path of the first connector member, indicated by a circumferential line, the first connector members has a tapered nose region extending in an axially convergent manner into the second female connector member which tapers forwardly to receive the tapered, axially convergent nose of the first member with a corresponding slope that results in a snug fit between the first and second connector members. The inner diameter of the second connector member is at least as great as the inner diameter of the first connector member at its nose portion 55 so that the second connector member acts as a sheath for the first member while preserving the integrity of the axial flow path. The second connector member has a cylindrical non-tapering forward portion, the male outlet 21, with opposed Luer lugs 81 and 83 at the forwardmost region of the second connector member, forming the Luer outlet fitting 31, shown in FIG. 4, at a forward end of the second tubular connector member 19. The Luer lugs 81 and 83 will fit into a male conical lock fitting, not shown, associated with an end of medical tubing. Returning to FIG. 3, an optional cup shaped rubber gasket 85, with a radially inwardly extending lip can be placed between the first and second connector members, slightly separating the two members but allowing axial flow. The radially inwardly extending rim of the gasket fits into a circumferential groove in the second flange 18 on the inward side of the latch members 42 and 44, while the radially outwardly extending rim of the gasket fits into a circumferential groove in the second flange 18 on the outward side of the latch members 42 and 44.

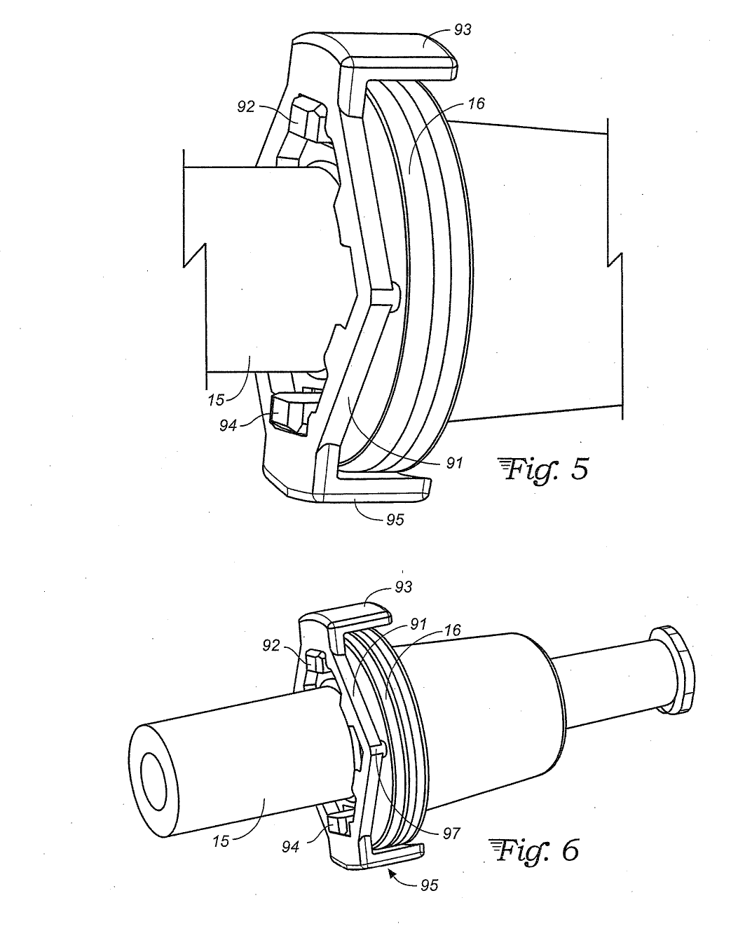

FIGS. 5 and 6 show a third connector member that is a deformable, mostly flat pivot ring 91 mounted about the outer diameter of the tubular male port 15 rearward of the first flange 16. The pivot ring has finger pad sized opposed extenders 93 and 95 parallel to the axis with opposed radial portions riding on the latch ends 92 and 94 and diametrically opposed pivots in the pivot holes. Fingers of a human hand can compress the opposed axial extenders 93 and 95 causing compression of opposed radial regions of the pivot ring by deformation of the third member about the pivots 97, releasing the opposed latch ends 92 and 94 to retreat through the first flange 16 thereby causing separation of the first connector member from the second connector member.

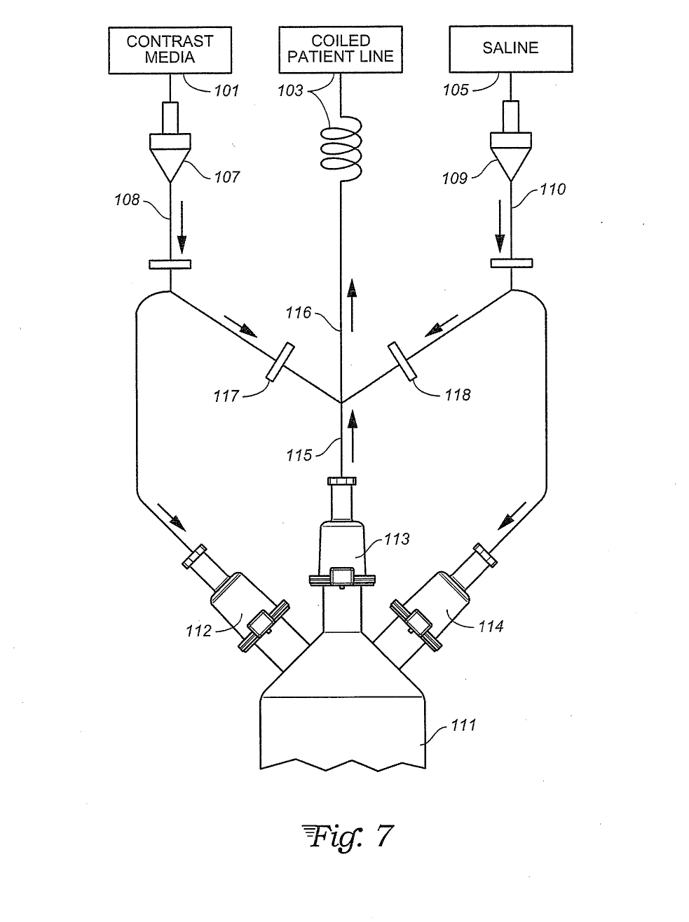

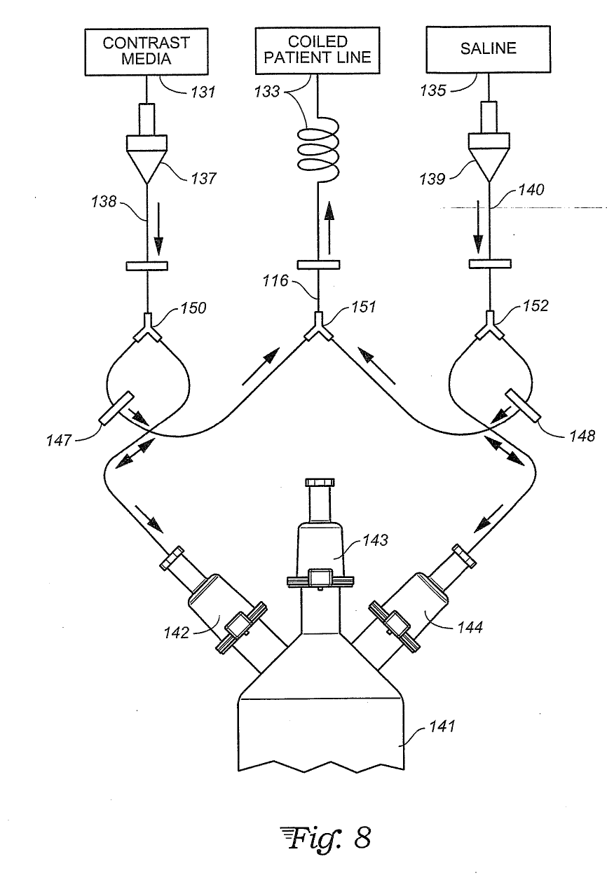

With reference to FIGS. 7 and 8, two possible scenarios for use with a patient connect a syringe, as well as contrast media and saline bags through connector members of the present invention and several check-valves and optional flow splitters to a coiled patient line of sterile medical tubing. In particular, in FIG. 7, a contrast media bag 101 connects via a connector 107 to sterile medical tubing 108. Likewise, a saline bag 105 connects via another connector 109 to sterile medical tubing 110. Both tubes 108 and 110 are preferably supplied with check-valves. A syringe 111 has multiple connectors 112-114 associated with it, wherein connectors 112 and 114 are directed for flow input to the syringe head and connector 113 is directed for flow output from the syringe head. Thus, the syringe 111 receives a portion of the contrast media and saline from respective bags 101 and 105 for pre-mixing with medication being delivered by the syringe 111 through connector 113. The remainder of the contrast media and saline may be delivered through tubing with check-valves 117 and 118 to combine directly with the sterile medical tubing 115 and 116. The combined mixture is then delivered through coiled patient line 103 to a patient. In FIG. 7, many of the tubes connect directly to each other. In FIG. 8, T- or Y-flow splitting connectors 150-152 for the tubing are expressly shown. FIG. 8 also has a different arrangement, wherein connector 142 on the syringe head 141 is for flow input receiving contrast media, connector 144 is bi-directional for alternately receiving input saline, premixing, and then delivering outward flow of the mixture. The third available connector 143, not being needed in this configuration, is sealed shut. Hence, contrast media bag 131 and saline bag 135 connect via connectors 137 and 139 to respective sterile medical tubes 138 and 140 leading to flow splitters 150 and 152. A portion of the material is delivered directly, via check-valves 147 and 148, to flow splitting connector 151, which in this case operates as a flow combiner into sterile medical tubing 116 and then through a check-valve to the coiled patient line 133. Other possible arrangements of medical syringes and media bags, sterile medical tubing and various numbers of connector devices of the present invention may be constructed as needed. Check-valves are placed where needed to limit flow and prevent over-pressure situations that could adversely affect a patient.

What is claimed is:

1. A connector for medical devices comprising:
a first tubular connector member extending from a medical device with an outward end and having an axial flow path for fluids with a first connector body having a first flange rearward of the outward end and having a nose end that tapers radially inwardly toward the outward end:
a second tubular connector member coaxial with the first'tubular connector member with a portion of the second tubular member having an axially divergent shape acting as a sheath for the nose end of the first tubular connector with a first end that surrounds a portion of the first tubular connector member including a second flange forward of the first end and abutting the first flange of the first connector member, the second flange having projecting latch members with flexing endwise teeth that project rearwardly through corresponding holes in the first flange whereby the first tubular connector member latches to the second tubular connector member in a secure manner.

2. The apparatus of claim 1 further comprising an annular release member that is coaxial with the first tubular connector member and joined to the first flange at opposed pivot holes that are spaced apart from the latch members, the release member having finger tabs near the latch members allowing human finger to flex the release member causing the latch members to move in a direction allowing reverse passage through the first flange of the first connector member when the first and second connector members are pulled in opposite directions, whereby separation of the second tubular connector member from the first connector member is possible.

3. The apparatus of claim 1 wherein the first connector member has a nose region tapering axially convergently forward of the first flange and wherein the second connector member has a corresponding taper along a length of the nose region and then extends forwardly of the nose region to provide a continuous fluid flow path in the axial direction.

4. The apparatus of claim 3 wherein the forwardmost region of the second connector member terminates in Luer lugs.

5. The apparatus of claim 2 wherein the second connector member has a separable annular end cap with a circumferential rim that fits into a circumferential detent in the nose region of the first connector member, whereby separation of the first and second connector members leaves the end cap with the first connector member, preventing joinder of a different second connector member without removal of the end cap.
